(19) United States
(12) Patent Application Publication
(10) Pub. No.: US 2006/0172953 A1
(43) Pub. Date: Aug. 3, 2006

(54) Treatment of benign prostatic hyperplasia using energolytic agents

(75) Inventors: George Tidmarsh, Portola Valley, CA (US); Harold E Selick, Belmont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Threshold Pharmaceuticals Inc., Redwood City (US)

(21) Appl. No.: 10/542,312

(22) PCT Filed: Jan. 16, 2004
(86) PCT No.: PCT/US04/01146

§ 371 Date: Feb. 7, 2006

Related U.S. Application Data
(60) Provisional application No. 60/441,110, filed on Jan. 17, 2003.
(60) Provisional application No. 60/442,344, filed on Jan. 23, 2003.
(60) Provisional application No. 60/458,663, filed on Mar. 28, 2003.
(60) Provisional application No. 60/458,665, filed on Mar. 28, 2003.
(60) Provisional application No. 60/458,846, filed on Mar. 28, 2003.
(60) Provisional application No. 60/460,012, filed on Apr. 2, 2003.
(60) Provisional application No. 60/472,907, filed on May 22, 2003.
(60) Provisional application No. 60/488,265, filed on Jul. 18, 2003.
(60) Provisional application No. 60/496,163, filed on Aug. 18, 2003.

(51) Int. Cl. A61K 31/70; A61K 31/445; A61K 31/19; A61K 31/11; G01N 33/574

(52) U.S. Cl. 514/23; 514/557; 514/317; 514/700; 435/7.23

(57) ABSTRACT

The invention provides a method for treatment or prophylaxis of benign prostatic hyperplasia by administration of an agent that interferes with energy metabolism, particularly the production of ATP and NADH/NADPH, in prostate epithelial cells.

### CROSS-REFERENCE TO RELATED APPLICATIONS

This application claims benefit of U.S. provisional application No. 60/496,163 (filed 18 Aug. 2003), 60/488,265 (filed Jul. 18, 2003), 60/472,907 (filed 22 May 2003), 60/460,012 (filed 2 Apr. 2003), 60/458,846 (filed 28 Mar. 2003), 60/458,665 (filed 28 Mar. 2003), 60/458,663 (filed 28 Mar. 2003), 60/442,344 (filed 23 Jan. 2003), and 60/441,110 (filed 17 Jan. 2003), each of which is incorporated herein by reference in its entirety for all purposes.

### FIELD OF THE INVENTION

The invention relates to treatment and prevention of benign prostatic hyperplasia and has application in the field of medicine and allied fields including but not limited to chemistry, medicinal chemistry, and biology.

### BACKGROUND OF THE INVENTION

Benign Prostatic Hyperplasia (BPH), a disease in which prostate epithelial cells grow abnormally and block urine flow, afflicts more than 10 million adult males in the United States, and many millions more throughout the rest of the world. Until relatively recently, surgical intervention was the only treatment of the disease, and even today, surgery is the treatment of last resort, almost inevitably relied upon when other treatments are or cease to be effective. Prostate surgery and recovery therefrom is painful, and the surgery itself may not be effective and poses the risk of serious side effects. For a recent review of the role of surgery in the treatment of BPH, see Barry, 2001 (full citations are provided below).

Only two classes of drugs are currently available to treat the symptoms of BPH. One class includes compounds that inhibit production of the active form of testosterone (dihyrdotestosterone or DHT). Use of these drugs can cause a loss of libido and loss of muscle mass and tone in males and is associated with an increased occurrence of high grade prostate cancer. In addition, this therapy is limited by the very long delay (months) between first administration of the drug and significant reduction in prostate size. The second class of currently used drugs for BPH, alpha adrenergic blockers, relaxes the smooth muscles, allowing urine to pass through the urethra more freely. While this class of drugs reduces symptoms more rapidly than the first, it does not reduce the size of the prostate or prevent it from growing larger, which can lead to eventual surgical intervention.

Thus, there is a significant, unmet need for drugs that can treat the underlying disease condition of BPH without serious side effects. The present invention meets that need.

### SUMMARY OF THE INVENTION

The present invention provides methods and compositions for treating BPH by administration of a compound (an “energolytic agent”) that inhibits glycolysis, impairs mitochondrial function or otherwise interferes with energy metabolism in prostate epithelial cells. The methods of the invention can be practiced using any glycolytic inhibitor that inhibits glycolysis in prostate epithelial cells or compound that impairs energy production or mitochondrial function in those cells. Illustrative classes of such compounds include, without limitation, a compound that inhibits glycolysis directly or indirectly, a compound that interferes with energy metabolism, a compound that impairs mitochondrial function, a mitochondrial poison, a glycolytic inhibitor, an inhibitor of hexokinase, lonidamine or a lonidamine analog, gossypol or a gossypol analog, 3-bromopyruvate or an analog thereof, and 2-deoxyglucose (2DG) or a 2DG analog. In addition, agents that directly or indirectly interfere with expression of HIF-1α, (thereby reducing glucose uptake by prostate epithelial cells) can be used in accordance with the methods of the invention.

Thus, in one aspect, the invention provides a method for treating benign prostatic hypertrophy (BPH) by administering a therapeutically effective amount of an agent that interferes with energy metabolism in prostate epithelial cells (an “energolytic agent”) to a human subject in need of such treatment.

In a related method, the invention provides a method for reducing a symptom associated with BPH by administering a therapeutically effective amount of an energolytic agent to a human subject exhibiting the symptom.

In a related method, the invention provides a method for reducing prostate size in a human subject by administering a therapeutically effective amount of an energolytic agent to the subject

In a related method, the invention provides a method for prophylaxis of BPH by administering a prophylactically effective amount of an energolytic agent to a human subject.

In some embodiments, the energolytic agent is selected from the group of 2-deoxyglucose, 3-bromopyruvate, gossypol, oxamate, iodoacetate, apoptolidin, londamine, an analog of 2-deoxyglucose, 3-bromopyruvate, gossypol, oxamate, iodoacetate, apoptolidin, and londamine.

In some embodiments of the invention, the subject is neither diagnosed with nor under treatment for cancer; and/or has a serum PSA greater than about 2 ng/ml; and or has a serum PSA less than about 10 ng/ml; and/or has previously been treated for BPH.

In some embodiments, the energolytic agent is administered in combination with another treatment for BPH. The other treatment for BPH can be, for example, administration of a second agent that interferes with energy metabolism in prostate epithelial cells, prostate reduction surgery, and/or administration of a drug from one of the two classes of drugs currently used to treat BPH.

In one embodiment, the energolytic agent is administered at least once daily for at least five days. In one aspect of the invention, the subject's AUASI or IPSS score is decreased by at least 3 points, optionally by at least about 5 points; prostate size has decreased by at least about 20%, optionally at least about 40%; and/or serum PSA levels are decreased by at least about 20%, optonally at least about 40%, when determined on or after 60 days after the initiation of treatment and compared to a baseline prior to the initiation of treatment.

The invention further provides a method for treating BPH by (a) diagnosing BPH in a patient, (b) administering an energolytic agent (EA) to the patient and (c) determining whether one or more manifestations of BPH are reduced in the patient. Also provided is a method for treating BPH by (a) administering an energolytic agent to a patient diagnosed with BPH and (b) determining whether one or more manifestations of BPH is reduced in the patient.

### BRIEF DESCRIPTION OF THE FIGURES

FIG. 1 shows structures for lonidamine (I, R═Cl), tolnidamine (I, R═CH₃), AF-2364 (II) and AF-2785 (III).

FIG. 2 shows structures of selected 2-DG analogs.

FIG. 3 shows the expression of HIF-1α in LNCaP cells under normoxic and hypoxic conditions and in the presence and absence of lonidamine. FIG. 3A shows an assay using a nuclear extract. FIGS. 3B and 3C show an assay using a whole cell extract.

FIG. 4 shows the expression of HIF-1α in PC-3 cells under normoxic and hypoxic conditions and in the presence and absence of lonidamine. FIGS. 4A and 4C show an assay using a nuclear extract. FIG. 4B shows an assay using a whole cell extract.

FIG. 5 shows lonidamine-induced apoptosis in LNCaP (FIG. 5A) and PC-3 (FIG. 5B) cells

FIG. 6 shows lonidamine-induced apoptosis in prostate epithelial cells.

FIG. 7 shows lonidamine-induced apoptosis in prostate epithelial cells (FIG. 7A) and prostate stromal cells (FIG. 7B).

FIG. 8 shows the effect of 0-600 μM lonidamine on expression of HIF-1α and other proteins as determined in whole cell extracts from LNCaP cells cultured under hypoxic conditions.

FIG. 9 shows the effect of 0-600 μM lonidamine on expression of HIF-1α and other proteins as determined in nuclear extracts from LNCaP cells cultured under hypoxic conditions.

### DETAILED DESCRIPTION OF THE INVENTION

### 1. Definitions

The following definitions are provided to aid in understanding the invention. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be assumed to represent a substantial difference over what is generally understood in the art.

As used herein, “treating” a condition or patient refers to taking steps to obtain beneficial or desired results, including clinical results. For purposes of this invention, beneficial or desired clinical results include, but are not limited to, alleviation or amelioration of one or more symptoms of BPH, diminishment of extent of disease, delay or slowing of disease progression, amelioration, palliation or stabilization of the disease state, and other beneficial results described below.

As used herein, “reduction” of a symptom or symptoms (and grammatical equivalents of this phrase) means decreasing of the severity or frequency of the symptom(s), or eliminating the symptom(s).

As used herein, “administering” or “administration of” a drug to a subject (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

As used herein, a “manifestation” of BPH refers to a symptom, sign, anatomical state (e.g., prostate size), physiological state (e.g., PSA level), or report (e.g., AUASI score) characteristic of a subject with BPH.

As used herein, a “therapeutically effective amount” of a drug is an amount of a drug that, when administered to a subject with BPH, will have the intended therapeutic effect, e.g., alleviation, amelioration, palliation or elimination of one or more manifestations of BPH in the subject. The full therapeutic effect does not necessarily occur by administration of one dose, and may occur only after administration of a series of doses. Thus, a therapeutically effective amount may be administered in one or more administrations.

As used herein, a “prophylactically effective amount” of a drug is an amount of a drug that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose, and may occur only after administration of a series of doses. Thus, a prophylactically effective amount may be administered in one or more administrations.

As used herein, “TID” and “QD” have their ordinary meanings of “three times a day” and “once daily,” respectively.

### 2. Benign Prostatic Hyperplasia and the Effects of Metabolic Inhibitors

The present invention provides compositions and methods useful in the treatment of benign prostatic hyperplasia (BPH). In particular, the invention relates to the use of compounds that inhibit or impair energy production in prostate epithelial cells for the treatment or prevention of BPH.

A brief discussion of the characteristics of BPH (also referred to as benign prostatic hyperplasia) will aid in the understanding of the invention. BPH involves overgrowth (hyperplasia) of cells in the prostate, resulting in enlargement of the prostate and leading to lower urinary tract symptoms and disease. The prostate gland contains secretory epithelial cells in a stroma of connective tissue and smooth muscle (see Barry, 2003, for a more detailed description of prostate anatomy), and BPH involves hyperplasia of the epithelial component. The secretory epithelial component in the normal prostate is remarkable in that the level of zinc in this tissue is exceedingly high compared to other normal tissues. A consequence of the high zinc levels is that, through a mechanism involving zinc inhibition of the enzyme m-aconitase, the generation of energy via the tricarboxylic acid (TCA) cycle and oxidative phosphorylation is substantially reduced in the secretory epithelium, making this tissue far more dependent than other organs and tissues upon glycolysis as an energy source. The zinc inhibition of m-aconitase, a key enzyme in the TCA cycle, results in at least a substantial reduction in, and perhaps a near complete blockade, of the TCA cycle in prostate epithelial cells. Another physiological result of the zinc-based inhibition of m-aconitase is the diversion of citrate from the TCA cycle, enabling the prostate to secret large quantities of citrate, used by the sperm as an energy source, into the seminal fluid. See, generally, Costello, 1999; Costello et al., 2000; Costello and Franklin, 2000.

As other normal cells in the body do not accumulate zinc to a level inhibitory to the metabolism of citrate, prostate epithelial cells are uniquely dependent on glycolysis (anaerobic metabolism) and so uniquely susceptible to inhibitors of glycolysis and to compounds that increase the near complete blockade of the tricarboxylic acid cycle in those cells, including but not limited to compounds that interfere with ATP and/or NADH/NADPH production, compounds that decrease glucose transport, and HIF-1α inhibitors. The methods of the invention target this increased reliance on glycolysis and the near complete blockage of the tricarboxylic acid cycle in prostate epithelial cells. Further, because the generation of energy via the tricarboxylic acid (TCA) cycle and oxidative phosphorylation is substantially reduced in the secretory epithelium (but probably not completely abolished) agents that impair mitochondrial function can also have differential effects in prostate epithelial cells. Without intending to be bound by a specific mechanism, it is believed that administration of agents that inhibit glycolysis and/or mitochondrial function in prostate epithelial cells preferentially starves these cells, relative to other cells, of energy. Without intending to be bound by a specific mechanism, it is believed that, by preferentially destroying the citrate producing cells by inhibiting glycolysis or by further impairing mitochondrial function, or both, enough of the hyperplastic, citrate-producing cells associated with BPH are destroyed to reduce the size of the prostate and relieve the condition and its clinical consequences. Thus, in accordance with the methods of the invention, a compound that inhibits or impairs energy production in prostate epithelial cells is administered to a human or other mammal with, or susceptible to, BPH at a dose that impairs energy production (decreases ATP levels) for a period of time that results in the preferential destruction of at least some of the citrate producing cells by starving them, relative to the normal cells in the body, of energy.

A compound that inhibits or impairs energy production in prostate epithelial cells is referred to herein as an “energolytic agent” or “EA.” It will be apparent to the reader that energolytic agents useful in the practice of the invention include compounds that inhibit glycolysis, compounds that impair mitochondrial function, and compounds that do both, including, in all cases, compounds that act directly or indirectly on glucose metabolism in the prostate. Thus, in one embodiment, the energolytic agent is a compound that impairs glycolysis in prostate epithelial cells. In one embodiment, the energolytic agent is a compound that impairs mitochondrial function in prostate epithelial cells. In one embodiment, the energolytic agent interferes with both glycolysis and mitochondrial function. In one embodiment, a combination of agents is used, including, in one embodiment, the administration of one agent that is an inhibitor of glycolysis and simultaneous or contemporaneous administration of a second agent that is an inhibitor of mitochondrial function.

One class of energolytic agents includes compounds that inhibit glycolysis (directly or indirectly). For example, the EA may inhibit an enzyme that catalyses a step in the conversion of glucose to pyruvate, or the oxidation of pyruvate to acetyl-CoA. For example, and not for limitation, the energolytic agent may be an inhibitor of hexokinase, glucokinase, phosphofructokinase, aldose, phosphoglycerate kinase, enolase, pyruvate kinase, pyruvate dehydrogenase. For illustration and not limitation such compounds include those described in U.S. Pat. No. 5,824,665 (6-amino-6-deoxy-glucose; N-acetyl-β-D-mannosamine; D-mannosamine; N-α-(p-tosyl)-L-lysine chloromethyl ketone); phosphoglycerate; quinone methides; taxodone; taxodione; α-methylene lactones; euparotin acetate; eupacunin; vernolepin; argaric acid; quinaldic acid; 5′-p-flurosuflonylbenzoyl adenosine; 5-keto-D-fructose; 5-keto-D-fructose-1,6-bisphosphate; Mg-phosphoglycerate; 2,3-diphosphoglycerate; 3(trans)-chlorophosphoenolpyruvate; 3(cis)-cyanophosphoenolpyruvate; D-tartronate; semialdehyde phosphate; aminoenolpyruvate; D-glycidol phosphate; L-glycidol phosphate; hydroxy-1-cyclopropanecarboxylic acid; D(−)3-phosphoglyceric acid; glyoxylate; hydroxypyruvate; kynurenate; xanthurenate; α-cyano-4-hydroxycinnamic acid; bromopyruvic acid; fluropyruvic acid) or pharmaceutically acceptable analogs or derivatives thereof. See: Bisswanger, 1981; Furuta, 1982; Waymack, 1979; Lowe, 1984; Bisswanger, 1980; Colombo, 1975; Hanson, 1970; McCune, 1989; Mansour, 1978; Avigad, 1974; Scopes, 1982; Gunter, 1982; Liu, 1990; Wirsching, 1985; Spring, 1971; Rose, 1969; O'Leary, 1981; de Domenech, 1980; and Johnson, 1982.

Another class of energolytic agents includes compounds that impair mitochondrial function (e.g., a mitochondrial poison). Mitochondrial poisons include but are not limited to the mitochondrial poisons described in U.S. Pat. No. 6,670,330.

As noted above, some energolytic agents may interfere with both glycolysis and mitochondrial function. For example, and without intending to limit the invention to a particular mechanism of action, the drug lonidamine disrupts the mitochondrial membrane, resulting in reduced activity of mitochondrially-bound hexokinase and interference with ATP production by the glycolytic pathway and oxidative phosphorylation. It will also be appreciated that agents that impair glycolysis will generally also at least indirectly reduce energy production by mitochondria, by reducing the amount of pyruvate available for entry into the TCA cycle.

Several exemplary energolytic agents are discussed below.

### 2-Deoxyglucose and Analogs of 2-Deoxyglucose

One energolytic agent suitable for use in the methods of the present invention is 2-deoxy-D-glucose (2-DG). 2-DG is phosphorylated by hexokinase to produce 2-DG-6-phosphate, which is not further metabolized and which inhibits hexokinase. 2-DG has been shown to inhibit glycolysis in cancer cells.

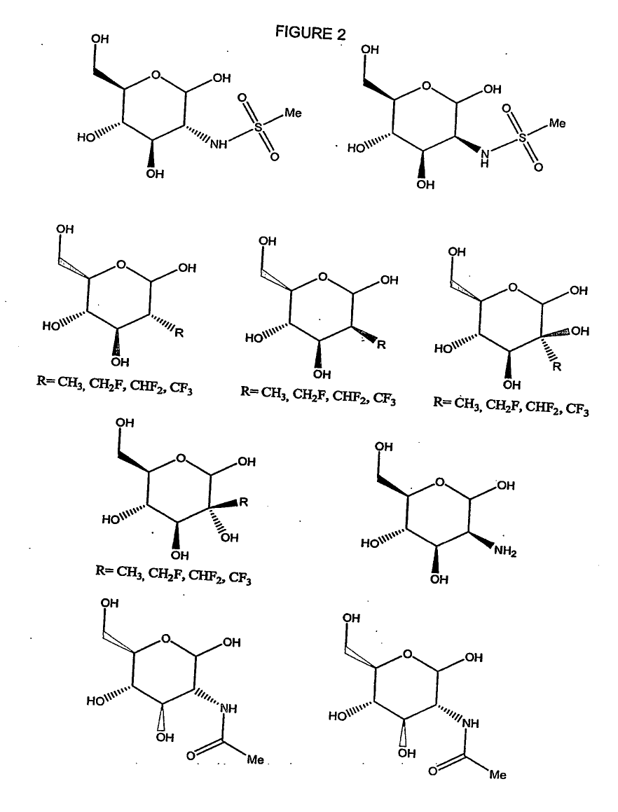

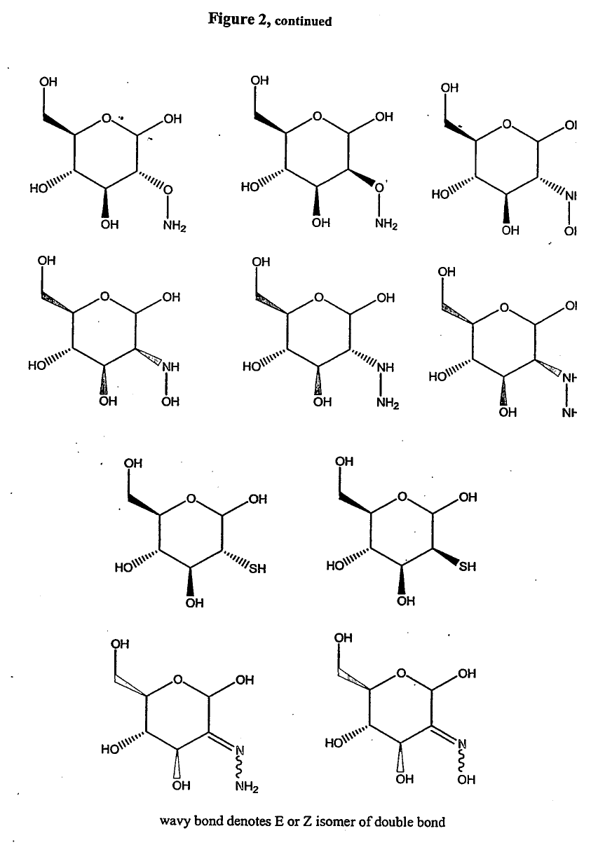

Another example of an energolytic agent is an analog of 2-DG that has glycolysis inhibiting activity. As used herein, a 2-DG analog is any D-glucose analog other than 2-DG that does not have a hydroxyl group at the 2 position of the glucose ring. L-glucose and its L-analogs are not 2-DG analogs for purposes of the present invention. A glucose analog includes mannose, galactose, glucose, and 5-thio-glucose. An analog of glucose or 2-DG can have a fluorine in place of a hydrogen at any position on the glucose ring; thus, 2-fluoro-2-deoxy-D-glucose (2-FDG) and 2-difluoro-2-deoxy-D-glucose are 2-DG analogs. An analog of glucose or 2-DG can have an amino group in place of a hydroxyl group at any position on the glucose ring other than the 6 position; thus, 2-amino-2-deoxy-D-glucose (2-glucosamine) and 2-amino-2-deoxy-D-galactose (2-galactosamine) are 2-DG analogs. Other illustrative 2-DG analogs include 2-F-mannose, 2-mannosamine, 2-deoxygalactose, 2-F-deoxygalactose, and di, tri, and other oligosaccharades that contain one or more of the preceding or following 2-DG analogs. Other 2-DG analogs useful in the methods of the present invention include the analogs shown in FIG. 2. 2-DG analogs useful in the present invention also include those analogs described in Reinhold, 2000, Oncol. Rep., 7:1093-97 (e.g., 2-deoxy-D-glucose tetraacetate) and in PCT publication WO 01/82926 (Lampidis, 2 Mar. 2001). Additional 2-DG analogs suitable for use in the methods of the present invention are described in U.S. patent application Ser. No. 10/______ (filed 9 Jan. 2004; attorney docket number 54492-2000400) entitled “Treatment Of Cancer With 2-Deoxyglucose.”.

### 3-Bromopyruvate and its Analogs

Another class of an energolytic agents suitable for use in the methods of the present invention is the class of 3-halo-pyruvates, including but not limited to 3-bromopyruvate, an inhibitor of hexokinase. For additional information on 3-halo-pyruvates, see U.S. Pat. No. 6,670,330 and U.S. patent application publication No. 20030087961.

Gossypol and Gossypol Analogs

Another class of energolytic agents sutiable for use in the methods of the present invention is the class composed of gossypol and its analogs, including but not limited to gossypol-(+), gossypol-(−), mixtures of gossypol-(+) and -(−), gossypol acetic acid, gossypol aldehyde, gossypol hemiacetal, gossypol quinoid, gossypolone, metabolites thereof, and physiologically acceptable salts thereof. Gossypol, gossypol analogs, formulations, and unit dose forms that can be employed in the methods of the present invention are described in PCT patent publication Nos. WO 02/097053; WO 02147673; U.S. Pat. Nos. 6,114,397 and 4,381,298; and U.S. patent application publication No. 2002137801.

Lonidamine and Analogs of Lonidamine

Another example of a class of energolytic agents suitable for use in the methods of the present invention is the class composed of lonidamine (1-(2,4-dichlorobenzyl)-1H-indazole-3-carboxylic acid; Doridamina™; lonidamine is also referred to herein as LND; see U.S. Pat. No. 3,895,026) and its analogs. Lonidamine was first identified as an anti-spermatogenic agent, and subsequently approved for the treatment of breast, cervical, lung and prostate cancers, in a few countries in Europe. See Silvestrini, 1981; Gatto et al., 2002. LND's anticancer properties have been reported to result at least in part from disruption of the mitochondrial membrane, resulting in reduced activity of mitochondrially-bound hexokinase and interference with ATP production by the glycolytic pathway and oxidative phosphorylation. See, Floridi et al., 1981, Fanciulli et al., 1996; and Gatto, 2002. Also see Kaplan, 2000. Methods for treating BPH by the administration of lonidamine and its analogs is described in copending U.S. patent application Ser. No. 10/______ (entitled “Treatment Of Benign Prostatic Hypertrophy,” attorney docket no. 54492-2000100, filed Jan. 16, 2004).

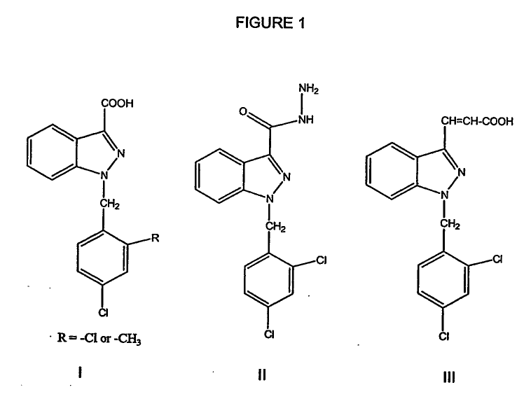

Examples of lonidamine analogs include, but are not limited to, tolnidamine; AF-2364, and AF-2785 (see FIG. 1; Ansari et al., 1998; and Corsi et al., 1976); compounds described by Silvestrini, 1981; Lobl et al., 1981, Cheng et al., 2001 and in U.S. Pat. Nos. 3,895,026 and 6,001,865.

Other Energolytic Agents

Other useful glycolytic inhibitors, mitochondrial function inhibitors, mitochondrial poisons, and hexokinase inhibitors useful in the methods of the present invention are known or can be identified using assays known in the art or described herein. For example, compounds for use as energolytic agents in the present invention include those described in PCT patent publication WO 01/82926 and U.S. Pat. Nos. 6,670,330; 6,218,435; 5,824,665; 5,652,273; and 5,643,883; and U.S. patent application publication Nos. 20030072814; 20020077300 (e.g., apoptolidin); and 20020035071.

Compounds for use as energolytic agents in the present invention also include the anti-metabolites described in U.S. patent publication No. 20020035071 (Pitha) including 3-O-methylglucose (Jay et al., 1990, J. Neurochem. 55: 989-1000); anhydrosugars such as 1,5-Anhydro-D-Glucitol (Polygalitrol) (Sols et al., 1954, J. Biol. Chem., 210:581-95; 1,5-anhydroglucitol-6-phosphate (Crane et al., 1954, J. Biol. Chem., 210:597-696; 2.5-Anhydro-D-Mannitol and 2,5-Anhydroglucitol)

Compounds for use as energolytic agents in the present invention also include inhibitors of lactate dehydrogenase, such as oxamate, and inhibitors of glyceraldehyde 3-phosphate dehydrogenase, such as iodoacetate (see, Lampidis, WO 01/82926).

Compounds that interfere with energy production in prostate epithelial cells and are useful for treatment of BPH and its symptoms can be identified by screens and assays known to those of skill in the art and/or described herein. For example, methods are known for identification of compounds that interfere with mitochondrial function, such as those described in U.S. Pat. Nos. 6,183,948 and 6,479,251. Assays for inhibitors of hexokinase (see Fanciulli et al., 1996, and Floridi et al., 1981) and other glycolytic enzymes are also known and can be used to screen for compounds suitable for use in the methods of the present as taught herein.

In addition, some agents useful in the practice of the invention are identified by their ability to mimic one or more activities of lonidamine, such as induction of apoptosis or inhibition of hypoxic induction of HIF-1α protein expression/accumulation in prostate epithelial cells or cell lines in vitro (e.g., inhibition of hypoxic induction of HIF-1α protein expression/accumulation). Assays and screens provided by the present invention to identify compounds with these properties are described below, in Section 7, and in the Examples.

Apoptosis assay in cell lines. As shown in Example 2, lonidamine induces apoptosis in cell lines derived from human prostate cells. The induction of apoptosis is significantly greater in LNCaP cells (ATCC NO. CLR-1740), a prostate-derived cell line that is citrate-producing, than in PC3 cells (ATCC NO. CLR-1435), a prostate-derived cell line that is citrate-oxidizing, consistent with the susceptibility of the citrate-producing prostate cells to metabolic inhibitors such as lonidamine. In some embodiments of the invention in which an energolytic agent is used for treatment or prevention of BPH or its manifestations, an agent with similar apoptosis-inducing activity is selected. Thus, in some embodiments of the invention, an energolytic agent that induces apoptosis (enhances caspase 3 activity) in citrate-producing prostate cells, such as LNCaP cells is administered to treat BPH. In some embodiments of the invention, an agent that induces apoptosis in LNCaP cells to a significantly greater degree than in PC3 cells is administered to treat BPH. In some embodiments of the invention, the induction of apoptosis by the agent is at least about 2-fold greater in LNCaP cells than in PC3 cells (and sometimes at least about 3-fold greater, at least about 4-fold greater, or at least about 10-fold greater) when assayed at the concentration of agent at which the difference in the level of apoptosis in the two cell lines is greatest (provided that the concentration of agent used in the assay is not greater than 1 mM).

Apoptosis assay in primary cell cultures. As shown in Example 2, lonidamine induces apoptosis in primary cultures of human prostate epithelial cells. The induction of apoptosis is significantly greater in primary cultures of prostate epithelial cells than in primary cultures of human prostate stromal cells, consistent with the susceptibility of citrate-producing prostate cells to metabolic inhibitors such as lonidamine. In some embodiments of the invention in which an energolytic agent is used for treatment or prevention of BPH or its manifestations, an agent with similar apoptosis-inducing activity is selected. Thus, in some embodiments of the invention, an energolytic agent for use in the invention induces apoptosis in prostate epithelial cells. In some embodiments of the invention, an agent that induces apoptosis in primary cultures of prostate epithelial cells to a significantly greater degree than in primary cultures of human prostate stromal cells is administered to treat BPH. In some embodiments of the invention, the agent does not significantly induce apoptosis in stromal cells. In some embodiments of the invention, induction of apoptosis by the agent is at least 2-fold greater in epithelial cells than in stromal cells (and sometimes at least 4-fold greater, sometimes at 10-fold greater, and sometimes at least 20-fold greater) when assayed at the concentration of analog at which the difference in the level of apoptosis in the two cell lines is greatest (provided that the concentration of analog used in the assay is not greater than 1 mM). In one embodiment, the present invention provides a method to identify for agents useful in the treatment of BPH, such method comprising the step of determining whether the agent induces apoptosis in prostate epithelial cells to a greater extent than it induces apoptosis in prostate stromal cells, and if such agent does induce apoptosis in prostate epithelial cells to a greater extent than it induces apoptosis in prostate stromal cells, then identifying such agent as an agent useful in the treatment of BPH. As described herein, a variety of assays, such as a caspase 3 assay, can be used to determine the induction of apoptosis.

HIF-1α expression assay. As shown in Example 1, lonidamine reduced HIF-1α expression/accumulation (as measured in the nuclear fraction) in a citrate-producing cell cultured under conditions of hypoxia by almost 2-fold at 200 micromolar and by more than 5 fold (i.e., more than 10-fold) at higher lonidamine concentrations. Thus, in some embodiments of the invention, an energolytic agent reduces HIF-1α expression (prevents HIF-1α accumulation) in LNCaP cells cultured under hypoxic conditions by at least about 2-fold, at least about 5-fold or at least about 10-fold compared to culture in the absence of lonidamine.

In the figures corresponding to Example 1, the effect of lonidamine on HIF-1α expression in prostate cells appears more pronounced in LNCaP cells than in PC3 cells cultured under hypoxic conditions (oxygen level <0.1%). Some lonidamine analogs useful for treatment of BPH according to the present invention may have a similar effect.

The results of these experiments do not definitively establish the mechanism or specificity of inhibition of HIF-1α by lonidamine. Lonidamine's effect on HIF-1α levels may be due entirely or in part to a general inhibition of protein synthesis, described as an activity of lonidamine by Floridi et al., 1985. Lonidamine's effect on HIF-1α levels could also be due entirely or in part to lonidamine's effect on oxygen utilization by mitochondria. Hagen et al., 2003, reported that HIF-1α is constitutively synthesized but degraded in the presence of oxygen. It is possible that, under hypoxic conditions, inhibition of mitochondrial respiration by lonidamine reduces oxygen consumption by mitochondria. This in turn could lead to enhanced activity of the oxygen-dependent enzyme, prolyl hydrolase, which plays a role in the HIF-1α degradation pathway.

In addition to in vitro assays such as those described above, energolytic agents can be evaluated in vivo for use in the methods of the invention. For example and without limitation, suitable assays include measurements of prostate function and activity, including in vivo measurements of prostate function and in vivo measurements of prostate size.

In vivo measurements of prostate function. The effect of a compound on prostate function, and, in particular, on respiration, can be assessed by monitoring prostate tissue metabolism (e.g., reduced ATP, citrate, and/or lactate production by the prostate in animals) following administration of the compound. Some energolytic agents useful in the present invention will detectably. ATP, citrate, and/or lactate levels can be monitored directly and/or indirectly in vivo in animals including humans, non-human primates and other mammals using techniques of magnetic resonance spectroscopy (MRS) or other methods. See, for example, Narayan and Kurhanewicz, 1992; Kurhanewicz et al., 1991; Thomas et al., 1990, for descriptions of MRS assays.

In vivo measurements of prostate size. The effect of compounds on prostate size can be assessed following administration of the compound using standard methods (for example, ultrasonography, for humans, and ultrasonography and/or comparison of organ weight in animals). Assays can be conducted in humans or, more usually, in healthy non-human animals or in monkey, dog, rat, or other animal models of BPH (see, Jeyaraj et al., 2000; Lee et al., 1998; Mariotti et al., 1982). Some energolytic agents useful in the present invention will detectably reduce prostate size.

Any of a variety of energolytic agents may be used for treatment of BPH. In one embodiment, the energolytic agent is an inhibitor of hexokinase. In one embodiment, the energolytic agent is an inhibitor of glucokinase. In one embodiment, the energolytic agent is an inhibitor of phosphofructokinase. In one embodiment, the energolytic agent is an inhibitor of aldose. In one embodiment, the energolytic agent is an inhibitor of phosphoglycerate kinase. In one embodiment, the energolytic agent is an inhibitor of enolase. In one embodiment, the energolytic agent is an inhibitor of pyruvate kinase. In one embodiment, the energolytic agent is an inhibitor of pyruvate dehydrogenase. In one embodiment, the energolytic agent is an inhibitor of lactate dehydrogenase. In one embodiment, the energolytic agent is an inhibitor of glyceraldehyde 3-phosphate dehydrogenase. In one embodiment, the energolytic agent is an inhibitor of glucose transport. In one embodiment, the energolytic agent reduces glucose transporter levels or prevents those levels from rising. In one embodiment, the energolytic agent is 2-deoxy-D-glucose (2-deoxyglucose or 2-DG). In one embodiment, the energolytic agent is an analog of 2-deoxyglucose. In one embodiment, the energolytic agent is 2-deoxy-D-glucose tetraacetate or 5-thio-glucose. In one embodiment, the energolytic agent is gossypol. In one embodiment, the energolytic agent is a gossypol analog. In one embodiment, the energolytic agent is 3-bromopyruvate. In one embodiment, the energolytic agent is a 3-bromopyruvate analog. In one embodiment, the energolytic agent is an analog of lonidamine. In one embodiment, the energolytic agent is lonidamine. In one embodiment, the energolytic agent is tolnidamine. In one embodiment, the energolytic agent is oxamate. In one embodiment, the energolytic agent is iodoacetate. In one embodiment, the energolytic agent is apoptolidin. In one embodiment, the energolytic agent is an analog of apoptolidin.

In one embodiment, the energolytic agent has a molecular weight less than 1000, optionally less than 500. In one embodiment, the energolytic agent is synthetic and does not occur in nature.

In one embodiment, the energolytic agent is other than an inhibitor of hexokinase. In one embodiment, the energolytic agent is other than an inhibitor of glucokinase. In one embodiment, the energolytic agent is other than an inhibitor of phosphofructokinase. In one embodiment, the energolytic agent is other than an inhibitor of aldose. In one embodiment, the energolytic agent is other than an inhibitor of phosphoglycerate kinase. In one embodiment, the energolytic agent is other than an inhibitor of enolase. In one embodiment, the energolytic agent is other than an inhibitor of pyruvate kinase. In one embodiment, the energolytic agent is other than an inhibitor of pyruvate dehydrogenase. In one embodiment, the energolytic agent is other than an inhibitor of lactate dehydrogenase. In one embodiment, the energolytic agent is other than an inhibitor of glyceraldehyde 3-phosphate dehydrogenase. In one embodiment, the energolytic agent is other than an inhibitor of glucose transport. In one embodiment, the energolytic agent does not reduce glucose transporter levels or prevent those levels from rising. In one embodiment, the energolytic agent is not a direct or indirect inhibitor of HIF-1α.

In one embodiment, the energolytic agent is other than 2-deoxyglucose. In one embodiment, the energolytic agent is other than an analog of 2-deoxyglucose. In one embodiment, the energolytic agent is other than 2-deoxy-D-glucose tetraacetate. In one embodiment, the energolytic agent is other than 5-thio-glucose. In one embodiment, the energolytic agent is other than gossypol. In one embodiment, the energolytic agent is other than a gossypol analog. In one embodiment, the energolytic agent is other than 3-bromopyruvate. In one embodiment, the energolytic agent is other than a 3-bromopyruvate analog. In one embodiment, the energolytic agent is other than lonidamine. In one embodiment, the energolytic agent is other than tolnidamine. In one embodiment, the energolytic agent is other than an analog of lonidamine. In one embodiment, the energolytic agent is other than an inhibitor of aconitase.

In one embodiment, the energolytic agent is other than oxamate. In one embodiment, the energolytic agent is other than iodoacetate. In one embodiment, the energolytic agent is other than apoptolidin. In one embodiment, the energolytic agent is other than analog of apoptolidin.

In some embodiments the energolytic agent is other than zinc or any other than any agent known for use for treatment of BPH on Jan. 1, 2004, without regard to whether or not the agent is known to inhibit glycolysis or impair mitochondrial function.

Although for illustration a wide variety of energolytic agents have been described herein, it will be appreciated that an energolytic agent suitable for use in according to the invention for treatment or prevention of BPH or alleviation of its symptoms will be pharmaceutically acceptable, i.e., will not be toxic to the subject at the doses and formulation administered, or the detrimental effects of any toxicity (e.g., side effects) associated with the agent will be outweighed by the benefit to the subject. Methods for identification and assessment of pharmaceutically acceptable agents are well known in the medical and pharmaceutical arts. For example, the therapeutic index (i.e., dose ratio of therapeutic effects to toxic effects, which can be expressed as the ED₅₀/LD₅₀ ratio) can be estimated using cell culture assays and animal studies. The data obtained from are used to formulate a range of dosage for human use. Pharmaceutical compositions which exhibit large therapeutic indices are preferred.

In certain embodiments, the energolytic agent is a pharmaceutically acceptable salt of a compound named above. Pharmaceutically acceptable salts include addition salts with acids, as well as the salts with bases. Salts with bases are, for example, alkali metal or alkaline earth metal salts, such as sodium, potassium, calcium or magnesium salts, or ammonium salts, such as those with ammonia or suitable organic amines, e.g. diethylamine, di-(2-hydroxyethyl)-amine or tri-(2-hydroxyethyl)-amine. Suitable acids for the formation of acid addition salts are, for example, mineral acids, such as hydrochloric, hydrobromic, sulphuric or phosphoric acid, or organic acids, such as organic sulphonic acids, for example, benzenesulphonic, 4-toluenesulphonic or methanesulphonic acid, and organic carboxylic acids, such as acetic, lactic, palmitic, stearic, malic, maleic, fumaric, tartaric, ascorbic or citric acid.

In certain embodiments, the energolytic agent is a prodrug of a compound named above, or a prodrug of a pharmaceutically active metabolite. Prodrug forms are known in the art and include of ester, amide and other derivatives of compounds listed above.

### 4. Patients for Whom Administration of an Energolytic Agent Provides Benefit

Accordingly, administration of an energolytic agent to a human subject diagnosed with, or exhibiting symptoms of, BPH provides benefits such as reduction of severity or frequency of one or more symptoms, reduction in prostate size or rate of enlargement, improvement in perceived quality of life, and reversion of other manifestations of BPH toward a more normal state. Further, administration of an energolytic agent to a human subject in need of prophylaxis for BPH provides benefits such as a reduction in likelihood that BPH will appear, reappear or progress in the subject. It will be apparent to the reader that the material below is organized into sections for convenience only, and disclosure in any organizational section is applicable to any aspect of the invention disclosed herein.

In one aspect of the invention, an energolytic agent is administered to a subject in need of treatment for BPH. As used herein, “a subject in need of treatment for BPH” is a man diagnosed with BPH. BPH can be diagnosed using art-known methods and criteria. The most common test is the digital rectal examination in which a physician determines whether the prostate is of a normal size and firmness. Other diagnostic assays include a urine flow rate test, determination of post void residual urine volume (e.g., by palpitation of the abdomen, drainage of residual urine, x-ray urogramography, or ultrasonography), moderate or severe symptom scores on the American Urologic Association Symptom Index (AUASI; Barry et al., 1992) or International Prostate Symptom Score (IPSS; Barry et al., 2001), and other tests known in the art.

Desired clinical results of treatment for BPH include, but are not limited to, alleviation or amelioration of one or more symptoms of BPH (see below), a reduction in prostate size (see below), a reduction in AUASI or IPSS scores compared to base line measurements-prior to commencement of therapy (for example, by 3 points or more, such as by 5 points or more), AUASI or IPSS scores less than 8, a reduction in serum PSA by at least about 20%, such as by at least about 40%, a serum PSA less than 4, such as less than 2, improvement in urodynamic parameters, and other desired results that will be recognized by a treating physician as indicative of a reduction in severity of BPH in a subject. An assessment of the response to treatment can be made at any time following the first administration of the drug. For example, an assessment is made about 30 days, about 60 days, or about 90 after beginning treatment. Alternatively, assessment can be made about 6, 12, 18, 24 or more months after beginning treatment. Alternatively, an assessment can be made less than about 30 days, about 30 days, about 60 days, or about 90 days after a course of treatment ends.

In a related aspect, an energolytic agent is administered to a human subject exhibiting a symptom associated with BPH to reduce the frequency or severity of the symptom. As used herein, “a symptom associated with BPH” refers to any one or more of the following symptoms: (1) urgency, (2) terminal dribbling of urine, (3) frequent urination, (4) nocturia, (5) a weak/slow stream of urine, (6) a sense of incomplete bladder emptying, (7) intermittency of urination, (8) straining to urinate, (9) dysuria, (10) hematuria, (11) acute urinary retention, (12) urinary tract infection, and (13) incontinence.

Administration of an energolytic agent according to the methods of the invention typically results in a reduction in severity, or elimination, of one or more of these symptoms; usually results in either a reduction in severity of, or elimination of, all of these symptoms; and often results in elimination of all of these symptoms.

In another related aspect, an energolytic agent is administered to reduce prostate size in a human subject in need of such reduction. As used herein, “a subject in need of reduction of prostate size” is a man having an enlarged prostate gland as determined by (1) imaging (e.g., ultrasonography, magnetic resonance imaging) and/or (2) one or more signs or symptoms resulting directly or indirectly from compression of the urethra by the prostate (e.g., including the symptoms of BPH discussed herein). A reduction in serum PSA (prostate specific antigen) is also a useful proxy for reduction of prostate volume. Although varying among individuals, enlarged prostates often exceed 30 grams, 40 grams, or 50 grams in size. The degree of reduction of prostate size will vary from subject to subject due to a number of factors, including the degree of enlargement at the time of onset of therapy, but will typically be a reduction of at least about 10% volume, more often at least about 25%, sometimes at least about 40%, sometimes at least about 50%, and sometimes an even greater than 50% reduction. in prostate size is observed. This reduction can be determined by imaging or other methods. Serum PSA can also in some instances serve as a useful proxy for prostate volume.

In a related aspect, an energolytic agent is administered to a subject with a serum PSA level greater than 2 ng/ml. PSA is secreted only by the epithelial cells of the prostate. For men with BPH, higher PSA levels suggest a relatively higher ratio of epithelial cell proliferation to stromal cell proliferation than in men with lower PSA levels. The present invention provides a number of diagnostic methods suitable for use in determining patients who should respond favorably to treatment with an energolytic agent. Thus, such treatment can provide a particularly good result in subjects with PSA levels greater than 2 ng/ml. Accordingly, subjects predicted to benefit most significantly from the methods of the invention can be selected in a population of men with BPH by identifying subjects with a serum PSA value greater than 2 ng/ml. In one embodiment of the invention, the subject has a PSA level greater than about 4 ng/ml. Because higher PSA levels are more closely associated with prostate cancer than with BPH, in one embodiment, the subject selected for therapy with an energolytic agent has a PSA level less than about 10 ng/ml.

In one aspect of the invention, an energolytic agent is administered to a subject who would benefit from prophylaxis of BPH. In one example, “a subject who would benefit from prophylaxis of BPH” is a man previously treated for BPH by surgery, transurethral microwave thermotherapy, transurethral needle ablation, transurethral electrovaporization, laser therapy, balloon dilatation, prostatic urethral stent, drug therapy, or other therapy and not currently diagnosed with or exhibiting symptoms of BPH. In another example, a subject who would benefit from prophylaxis of BPH is a man at increased risk for developing BPH due to age (e.g., men older than 40, older than 50, older than 60, or older than 70 years of age). In another example, a subject who would benefit from prophylaxis of BPH is a man who is asymptomatic, or has symptoms sufficiently mild so that no clear diagnosis of BPH can be made, but who has an elevated serum PSA level (e.g., PSA>2 ng/ml or, in some cases, >4 ng/ml).

It will be clear from the foregoing that, in some cases, the subject to whom an energolytic agent is administered is a man who has previously been treated for BPH, while in other cases the subject is a man who has not previously been treated for BPH.

In one embodiment of the invention, the subject in need of treatment or prophylaxis for BPH is not also under treatment for cancer. In a related embodiment, the subject in need of treatment or prophylaxis for BPH has not been diagnosed as having cancer. In one embodiment, the subject in need of treatment or prophylaxis for BPH does not have cancer. In one embodiment, the subject in need of treatment has a cancer other than prostate cancer but does not have prostate cancer. As used herein, “cancer” has its ordinary medical meaning and refers to a malignancy (including head, neck, prostate and breast cancers, leukemias and lymphomas), generally characterized by clonality, autonomy, anaplasia, and metastasis (see Mendelsohn, 1991).

In one embodiment, the invention provides a method of treating BPH in a patient by administering an energolytic agent to the patient. In a related embodiment, the invention provides a method for treating BPH comprising (a) administering an energolytic agent to a patient diagnosed with BPH and (b) determining whether one or more manifestations of BPH are reduced in the patient. In one embodiment, the invention provides a method for treating BPH by (a) diagnosing BPH in a patient, (b) administering an energolytic agent to the patient and (c) determining whether one or more manifestations of BPH are reduced in said patient. In the foregoing embodiments, optionally the subject is not diagnosed with or under treatment for cancer; optionally has a PSA>2 ng/ml, optionally has a PSA>2 ng/ml and <10 ng/ml.

In another aspect, the invention provides a method entailing (a) advertising the use of an energolytic agent for treatment of BPH, and (b) selling an energolytic agent to individuals for use for treatment of BPH. In one embodiment, the advertising makes reference to a trademark that identifies an energolytic agent product and the energolytic agent sold is identified by the same trademark. It will be appreciated that the individuals to whom an energolytic agent is sold include corporate persons (corporations) and the like and “selling BPH to individuals for use for treatment of BPH” includes selling to, for example, a medical facility for distribution to patients for treatment of BPH.

### 5. Dose, Route, Schedule and Duration of Administration

A variety of routes, dosage schedules, and dosage forms are appropriate for administration of energolytic agents for treatment and prophylaxis of BPH. Appropriate dosage schedules and modes of administration will be apparent to the ordinarily skilled practitioner reading the present disclosure and/or can be determined using routine pharmacological methods.

The dose, schedule and duration of administration of the energolytic agent will depend on a variety of factors. The primary factor, of course, is the choice of a specific agent. Other important factors include the age, weight and health of the subject, the severity of BPH symptoms, if any, the subject's medical history, co-treatments, goal (e.g., therapy or prophylaxis), preferred mode of administration of the drug, the formulation used, patient response to the drug, and the like. Guidance concerning administration is provided by prior experience using the agent for a different indication (e.g., lonidamine administered to treat cancer is administered in 150 mg doses three times a day for a period of about a month), and from new studies in humans and other mammals. Cell culture studies are frequently used in the art to optimize dosages and the assays disclosed herein can be used in determining such doses (e.g., to determine the dose that induces signification apoptosis in prostate epithelial cells but not in prostate stromal cells or other cells). For particular agents, the scientific literature (including, for example, patent and non-patent publications cited herein) provides considerable guidance as to dosages, formulations and dosage forms for specific agents or classes of agents, e.g., dosages known or predicted to result in a biologically effective serum level of the agent (or metabolite) in serum.

For example, an energolytic agent can be administered for the treatment of BPH at a dose in the range of about 1 mg to about 2 g of the energolytic agent per kg of body weight of the patient to be treated, with more than one dose being administered. In one embodiment, an energolytic agent is administered in a dose in the range of about 1 mg to about 5 per kg of body weight of the patient to be treated. In another embodiment, an energolytic agent is administered in a dose in the range of about 100 mg to about 1 g per kg of body weight of the patient to be treated. In certain other embodiments, an energolytic agent is administered in a dose of about 50 to 250 mg per kg of body weight of the patient to be treated. In another embodiment, the therapeutically effective dose is about 50 mg/kg to about 500 mg/kg. For illustration, the therapeutically effective dose of an energolytic-agent can be administered daily or once every other day or once a week to the patient. Generally, multiple administrations of the agent are employed. Depending on the dose selected by the practitioner and the convenience of the patient, the entire dose may be administered once daily, or the dose may be administered in multiple smaller doses through the course of a day. For example, the dose may be divided into two smaller doses and administered twice daily, or divided into three smaller doses and administered thrice daily. Alternatively, the dose may be combined and given every other day, or even less frequently, but in any event, the dose is repeatedly administered over a period of time. For optimum treatment benefit, the administration of the therapeutically effective dose is continued for multiple days, typically for at least five consecutive days, and often for at least a week and often for several weeks or more. In one embodiment, the energolytic agent is administered once (qday), twice (bid), three times (tid), or four times (qid) a day or once every other day (qod) or once a week (qweek), and treatment is continued for a period ranging from three days to two weeks or longer. In one embodiment, the treatment is continued for one to three months. In another embodiment, the treatment is continued for a year. Thus, a patient may be administered the energolytic agent for a week, a month, two months, three months, six months, or a year or longer. For preventive applications, treatment may continue indefinitely throughout the life of the patient. As is well understood in the medicine, treatment may be suspended temporarily if toxicity is observed or for the convenience of the patient without departing from the scope of the invention.

For illustration and not limitation, the present invention provides a pharmaceutical formulation of an energolytic agent suitable for oral administration (including tablets, capsules, and pills) and contains between 1 and 100 mg of the compound, and in another embodiment between 1 and 10 mg of the compound. In another embodiment, the formulation contains between 200 and 1000 mg of the compound, and in another embodiment between 500 and 1000 mg of the compound.

In addition, the present invention provides controlled and sustained release formulations of the compounds that allow once a day oral dosing. Such sustained release formulations (including tablets, capsules, and pills) of the invention contain between 1 mg and 3 g of the active compound, with various alternative embodiments, including one that contains between 1 mg and 10 mg of the compound; another that contains between 150 and 500 mg of the compound; and another that contains between 750 mg and 2 g of the compound.

In therapeutic and prophylactic applications, the energolytic agent can be administered a single time or many times over periods as long as several months or years. In one embodiment of the invention, the agent is administered to a symptomatic (e.g., experiencing difficulty in urination) BPH patient only until the symptoms abate or disappear, and then treatment is stopped unless and until symptoms reappear. When symptoms reappear, administration of the agent can be resumed. In another embodiment, treatment continues after symptoms disappear or are reduced to an acceptable target level, at least for a period of time, such as a week, two weeks, a month or several months. In another embodiment, the drug is administered to an asymptomatic subject to prevent the development or reoccurrence of symptoms (i.e., prophylactically administered). This time period may include continuous dosing TID for two to six months or more or for only one to eight weeks. A dose of 150 mg po TID for 7-30 days of certain agents of the invention, such as lonidamine and its analogs) can allow for the full therapeutic benefit in treating BPH while limiting or eliminating the unwanted side effects. In yet another embodiment, BPH is treated in accordance with the methods of the invention by administering to a BPH patient a much higher dose of a compound for a shorter period of time (that is, fewer administrations; in one embodiment, a single administration of a metabolic inhibitor is sufficient to provide relief from BPH symptoms).

When formulated for oral delivery, preferred dosage forms include pills, tablets, capsules, caplets, and the like, optionally formulated for sustained release. Other suitable forms for oral administration include troches, elixirs, suspensions, syrups, wafers, lozenges, and the like. Other modes of administration are also contemplated, including parenteral, inhalation spray, transdermal, rectal, intraprostetic injection (e.g., of EA-containing microparticles) and other routes.

In the case of lonidamine, exemplary dosage schedules are described in copending U.S. patent application Ser. No. 10/______ (entitled “Treatment Of Benign Prostatic Hypertrophy,” attorney docket no. 54492-2000100, filed Jan. 16, 2004). In one embodiment, the dosage form is the 150 mg unit dosage form marketed under the tradename Doridamina™ (e.g., 150 mg po TID for about thirty days). Other dosing regimens contemplated include, for example and not for limitation, “low dosing” (e.g., dosaged in the range of 1-300 mg per day total daily dosage, 5-300 mg/day, 5-70 mg/day, 1-25 mg/day, 20-45 mg/day, 40-65 mg/day, 40-70 mg/day, 50-100 mg/day, 50-200 mg/day, and 50-300 mg/day), “high dosing (e.g., total daily doses greater than 0.5 g, such as doses in the range 0.5-5 g/day, 0.5-3 g/day, 0.5-1 g/day and 1-3 g/day, or higher doses), and “intermediate dosing” (e.g., doses greater than 300 and less than 500 mg/day, such as doses in the range >300-400 or 400<500, e.g., 450 mg/day).

In the case of 2-deoxyglucose (2-DG) and analogs (2-DGA) thereof (e.g., such as those shown in FIG. 2), exemplary dosage schedules are described in copending U.S. patent application Ser. No. 10/______ (entitled “Treatment Of Cancer With 2-Deoxyglucose,” attorney docket no. 54492-20004.00, filed Jan. 9, 2004). For example, 2DG and 2DGA can be administered for the treatment of BPH at a dose in the range of about 1 mg to about 2 g of 2-DG or 2-DGA per kg of body weight of the patient to be treated. In another embodiment, 2-DG or a 2-DGA is administered in a dose in the range of about 10 mg to about 1 g of 2-DG or a 2-DGA per kg of body weight of the patient to be treated. In certain other embodiments, 2-DG or a 2-DGA is administered in a dose of about 50 to 250 mg of a 2-DG or a 2-DGA per kg of body weight of the patient to be treated. In another embodiment, the therapeutically effective dose is about 25 mg/kg to about 150 mg/kg. For illustration, the therapeutically effective dose of 2DG or a 2DGA is administered daily or once every other day or once a week to the patient, and multiple administrations of the drug are employed. Depending on the dose selected by the practitioner and the convenience of the patient, the entire dose may be administered once daily, or the dose may be administered in multiple smaller doses through the course of a day. For example, the dose may be divided into two smaller doses and administered twice daily, or divided into three smaller doses and administered thrice daily. Alternatively, the dose may be combined and given every other day, or even less frequently, but in any event, the dose is repeatedly administered over a period of time. For optimum treatment benefit, the administration of the therapeutically effective dose is continued for multiple days, typically for at least five consecutive days, and often for at least a week and often for several weeks or more. In one embodiment, 2DG or a 2DGA is administered once (qday), twice (bid), three times (tid), or four times (qid) a day or once every other day (qod) or once a week (qweek), and treatment is continued for a period ranging from three days to two weeks or longer. In one embodiment, the treatment is continued for one to three months. In another embodiment, the treatment is continued for a year. Thus, a, patient may be administered 2DG or a 2DGA for a week, a month, two months, three months, six months, or a year or longer. For preventive applications, treatment may continue indefinitely throughout the life of the patient. As is well understood in the medicine, treatment may be suspended temporarily if toxicity is observed or for the convenience of the patient without departing from the scope of the invention.

It will be appreciated that these dosing schedules are for illustration and not limitation, and that a dosing schedule may change during a course of therapy based on, for example, a patient's response to the therapy.

### 6. Treatment Combinations

Energolytic agents can be administered to a BPH patient in combination with other agents, including energolytic and non-energolytic agents, or procedures intended to treat BPH, ameliorate symptoms of BPH, potentiate the effects of the energolytic agent, or provide other therapeutic benefit. Administration of an agent “in combination with” includes parallel administration (administration of both the agents to the patient over a period of time, such as administration of an EA and tamsulosin on alternate days for one month), co-administration (in which the agents are administered at approximately the same time, e.g., within about 30 minutes of each other), and coformulation (in which the agents are combined or compounded into a single dosage form suitable for oral or parenteral administration). Exemplary agents for administration in combination with an energolytic agent (or a combination of energolytic agents, such as, for example, lonidamine and 2-DG) include, but are not limited to, zinc, alpha-blockers, 5-alpha-reductase inhibitors, and plant extracts (see below). As noted above, multiple different energolytic agents can be used in combination.

Zinc: As discussed above, high concentrations of zinc in the secretory epithelial cells of the prostate inhibit m-aconitase, increasing the dependence of that tissue on glycolysis for energy production. In -accordance with the methods of the present invention, it may in some patients be beneficial to co-administer zinc (e.g., zinc chloride, zinc gluconate, zinc sulfate, zinc acetate, zinc aspatate, zinc citrate, zinc glycerate, zinc oxide, zinc picolinate, and other zinc-containing compounds) with a drug composition of the invention, to maximize the efficacy of the treatment. For example and not limitation, 15-300 mg/day zinc can be administered, typically 30-50 mg/day).

Alpha-Adrenergic-Blockers: Alpha-blockers alleviate some symptoms of BPH, without curing the underlying disease. These agents work by relaxing the muscles at the neck of the bladder and in the prostate, reducing the pressure on the urethra. Exemplary alpha-blockers include doxazosin (Cardura), terazosin (Hytrin), tamsulosin (Flomax), alfuzosin (Xatral), and prazosin (Hypovase). In one embodiment of the invention, an alpha blocker is administered in combination with an energolytic agent to treat BPH. In another embodiment, the alpha-blocker is administered at a lower dosage (amount) or less frequently (e.g., alternate days rather than daily) than the “standard” dosage (the amount dosed in the absence of co-administration with an energolytic agent).

5-Alpha-Reductase Inhibitors: 5-alpha-reductase inhibitors inhibit the conversion of testosterone to dihydrotestosterone 2 (DHT), an androgen that contributes to prostate enlargement. An exemplary 5-alpha-reductase inhibitor is finasteride (Proscar). In one embodiment of the invention, a 5-alpha-reductase inhibitor is administered in combination with an energolytic agent to treat BPH. In another embodiment, the 5-alpha-reductase inhibitor is administered at a lower. dosage (amount) or less frequently (e.g., alternate days rather than daily) than the “standard” dosage.

Plants: Saw Palmetto (Serenoa repens) or an extract thereof, or Pygeum Africanum or an extract thereof can be administered in combination with an energolytic agent for therapeutic benefit in the treatment of BPH.

Procedures. In addition, an EA may be administered in combination with, or prior to, procedures for treatment of BPH including surgery (transurethral resection of the prostate; transurethral incision of the prostate; or open prostatectomy), laser therapy, transurethral microwave thermotherapy, balloon dilatation, placement of a prostatic urethral stent, transurethral needle ablation, transurethral electrovaporization of the prostate, or other non-rug therapies.

Hif-1α inhibitors. In some embodiments, an energolytic agent of the invention is administered to a BPH patient in combination with a Hif-1α inhibitor. Unless otherwise indicated, “Hif-1α” is used herein to refer to an agent causes a reduction in Hif-1α levels in a prostate cell, but does not specifically interfer with glycolysis or mitoichondrial function in the cell. Exemplary Hif-1α inhibitors include, but are not limited to P13 kinase inhibitors; LY294002; rapamycin; histone deacetylase inhibitors such as [(E)-(1S,4S,10S,21R)-7-[(Z)-ethylidene]4,21-diisopropy-2-oxa-12,13-dithia-5,8,20,23-tetraazabicyclo-[8,7,6]-tricos-16-ene-3,6,9,19,22-pentanone (FR901228, depsipeptide); heat shock protein 90 (Hsp90) inhibitors such as geldanamycin, 17-allylamino-geldanamycin (17-MG), and other geldanamycin analogs, and radicicol and radicicol derivatives such as KF58333; genistein; indanone; staurosporin; protein kinase-1 (MEK-1) inhibitors such as PD98059 (2′-amino-3′-methoxyflavone); PX-12 (1-methylpropyl 2-imidazolyl disulfide); pleurotin PX-478; quinoxaline 1,4-dioxides; sodium butyrate (NaB); sodium nitropurruside (SNP) and other NO donors; microtubule inhibitors such as novobiocin, panzem (2-methoxyestradiol or 2-ME2), vincristines, taxanes, epothilones, discodermolide, and derivatives of any of the foregoing; coumarins; barbituric and thiobarbituric acid analogs; camptothecins; and YC-1, a compound described in Biochem. Pharmacol., 15 Apr. 2001, 61(8):947-954, incorporated herein by reference, and its derivatives. In an aspect of the invention, a Hif-1α inhibitor can be administered alone (not in combination with an energolytic agent of the invention) to treat a subject with BPH.

### 7. Assays

In one aspect, the invention provides methods for determining the usefulness of a compound for treatment of BPH and methods for identifying compounds useful in the treatment of BPH. In one embodiment, the method involves (a) contacting a citrate-producing cell with the compound; (b) contacting a citrate-oxidizing cell with the compound; and (c) detecting a differential effect of said contacting on the citrate-producing cell compared to the citrate-oxidizing cell. A differential effect (e.g., as described herein) indicates that the agent may be useful for treatment of BPH. Further and confirmatory assays can then be conducted. The method can be conveniently carried out in vitro.

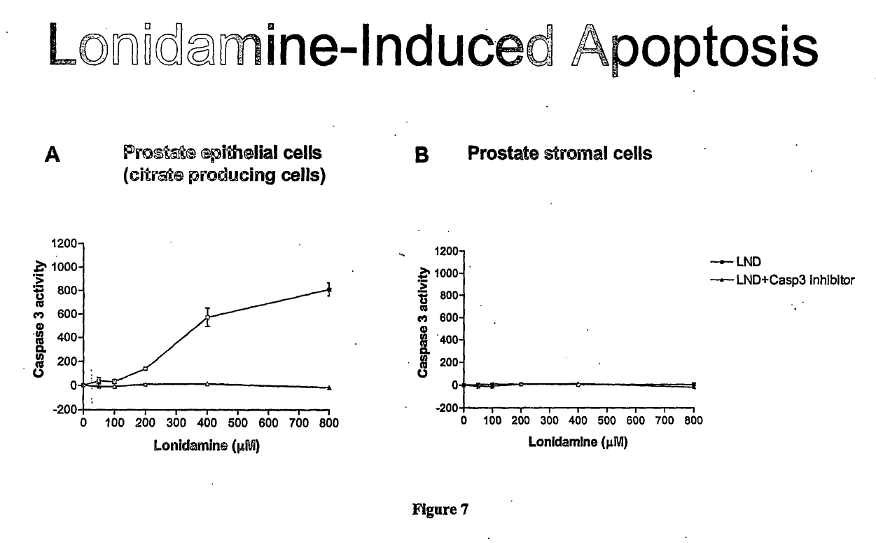

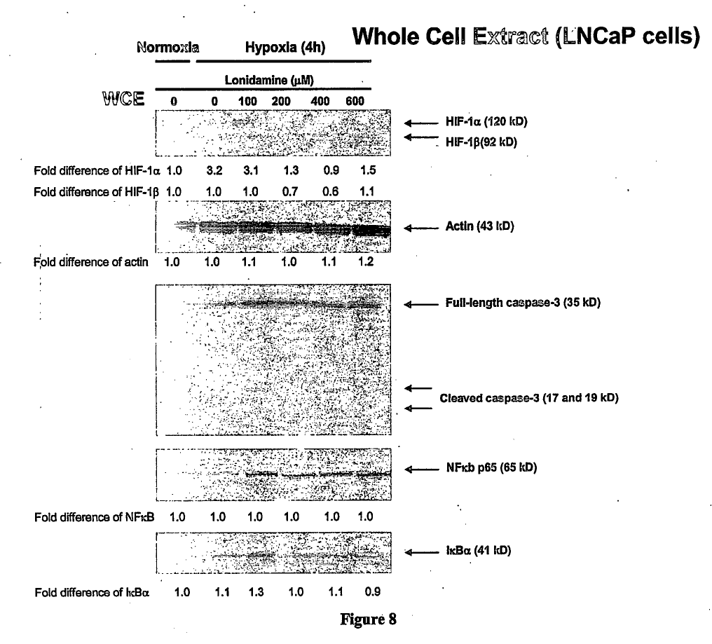

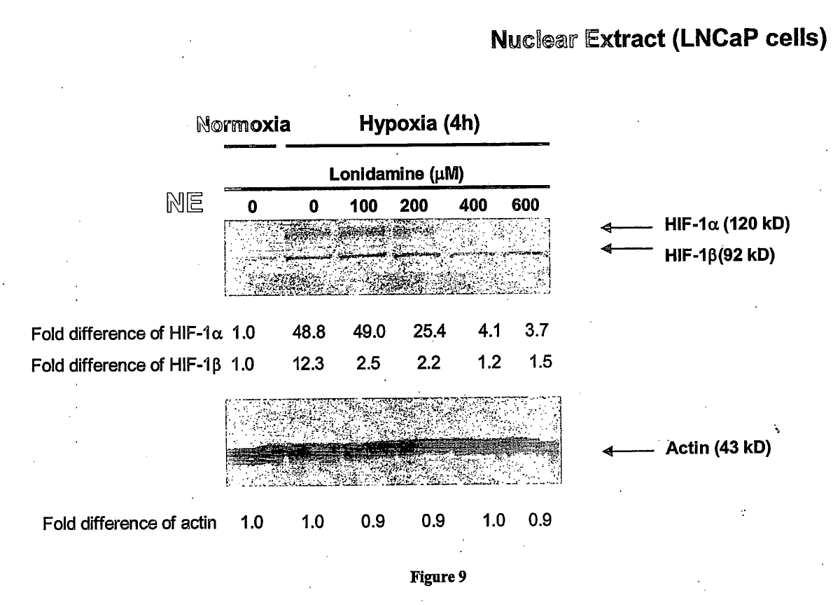

In a related aspect, the invention provides a method for determining the usefulness of a compound for treatment of BPH by (a) contacting a citrate-producing cell cultured under conditions of hypoxia with the compound; and (b) identifying a compound as useful for treatment of BPH if the contacting results in a dose-dependent reduction in HIF-1α expression (measured in the nuclear fraction) of at least about 2-fold, sometimes at least about 5-fold, and sometimes at least about 10-fold. A number of assays for HIF-1α expression are known. Generally, immunoassays (e.g., immunoblots and ELISAs) are most convenient. Reagents and methods for such assays are well known in the art. Expression of other proteins can be measured (see, e.g., FIG. 7) to obtain relative expression values.

These methods were developed, in part, based on the discovery that the energolytic agent lonidamine induces apoptosis of prostate cells, and that the induction is substantially more pronounced in citrate-producing cells compared to citrateoxidizing cells and that lonidamine reduces expression (accumulation) of HIF-1α in prostate cells, especially under hypoxic conditions.

A variety of cells and assays can be used in the methods of the invention. Citrate-producing and citrate-oxidizing cell types are known and can be identified using art-known assays and criteria. See, e.g., Costello and Franklin, 1997, Urology 50:3-12 and Franklin et al., 1995, Endocrine 3:603-607. Suitable citrate-producing cells include primary cultures of prostate epithelial cells and certain established cell lines derived from prostate epithelial cells (e.g., LNCaP cells). Suitable citrate-oxidizing cells include primary cultures of prostate stromal cells and certain established cell lines derived from prostate cells (many malignant prostate epithelial cells undergo an apparent metabolic transformation from citrate-producing to citrate-oxidizing; see Franklin et al.; 1995, Endocrine 3:603-607). In one embodiment, the citrate-producing cell is an LNCaP cell and the citrate oxidizing cell is a PC-3 cell. Primary cultures of human prostate epithelial and stromal cells are commercially available (e.g., cells can be obtained from Cambrex Bio Science Rockland, Inc., 191 Thomaston Street, Rockland, Me. 04841) and can be prepared according to well known tissue culture methods (see, e.g., Peehl, D M, Culture of Epithelial Cells: Prostate Culture, 1992, 159-180). Established cell lines derived from prostate are available from the American Type Culture Collection (ATCC), P.O. Box 1549, Manassas, Va. 20108 USA, or can be prepared according to well known methods. In another embodiment, the cell is selected from the group of recombinant cells selected from the group consisting of (i) a cell, optionally a cell other than a prostate cell, that has been modified so as to accumulate zinc to levels that inhibit m-aconitase and a cell other than a prostate cell that cannot metabolize citrate. In one embodiment, the cell is a cell that has been modified to not express m-aconitase or to express only an inactive mutant thereof. In one embodiment, the cells are human (although cells from other mammals also can be used). In one embodiment, the cells have been immortalized by modification such that telomerase expression occurs constitutively.

In a typical assay, cells are contacted with the test compound, usually at a range of concentrations (e.g., 10, 50,100, 200, 400, 600 and 800 μM). The contacting is conveniently achieved by adding the compound to the medium in which the cells are cultured, or any other method of contacting. In one embodiment, the compound is introduced into the cell or cell culture in a carrier (e.g., liposomal carrier) or solvent. It will be understood that, as is usual in drug screening assays, suitable controls (e.g., negative controls) and statistical methods are used. Assays can be carried out on whole cells, cell extracts or, alternatively, nuclear exctracts.

As is disclosed below in the Examples, it has been discovered that the some of the differential effects of lonidamine on citrate-producing and citrate oxidizing cells are most striking in cells grown under conditions of hypoxia. Accordingly, in some embodiments of the invention, the cells are grown under hypoxic conditions. For example, cells can be cultured in low oxygen levels (e.g., <0.1%). Hypoxia can also be induced by culture at high cell density.

An examples of a differential effect is induction of apoptosis that is greater in citrate-producing cells compared to citrate-oxidizing cells. In one embodiment the differential effect is induction of apoptosis that is greater in citrate-producing cells compared to citrate-oxidizing cells. In one embodiment, the differential effect is at least about 10-fold or at least about 20-fold. A number of assays for apoptosis or its markers or other indicators thereof are known and can be used in the present assay. For example and not limitation, apoptosis assays include assays for caspase 3; DNA fragmentation assays (e.g., TUNEL assays; BD Biosciences No 556381), and Annexin V assays (e.g. BD Biosciences No 556547).

In the figures corresponding to Example 1, the effect of lonidamine on HIF-1α expression in prostate cells appears more pronounced in LNCaP cells than in PC3 cells when cultured under hypoxic conditions (oxygen level <0.1%). Some energolytic agents useful for treatment of BPH according to the present invention may have a similar effect. Accordingly, another differential effect that can be measured is a reduction in HIF-1α expression that is greater in citrate-producing cells than in citrate-oxidizing cells, especially cells cultured under hypoxic conditions. For example, the difference is at least about 2-fold, and sometimes at least about 4-fold.

### 8. EXAMPLES

### Example 1

### Lonidamine Reduces Expression of HIF-1α in Prostate Cells

This example shows the effects of lonidamine treatment on HIF-1α expression in two cell lines derived from metastatic lesions of human prostate cancers. LNCaP is a citrate-producing cell (ATTC No. CRL-1740) while PC3 is citrate oxidizing cell (ATTC No.CRL-1435). See Franklin et al.; 1995, Endocrine 3:603-607. Cells may be obtained from the American Type Culture Collection (ATCC), P.O.Box 1549, Manassas, Va. 20108 USA.

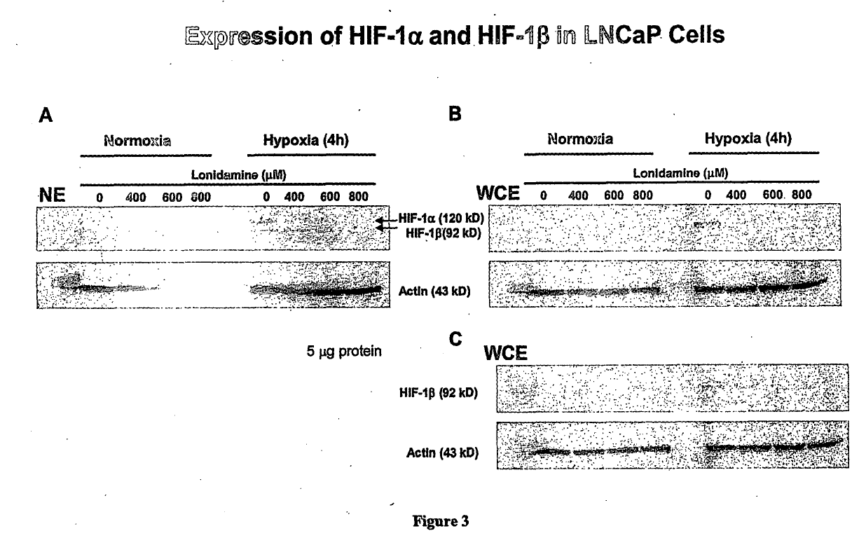

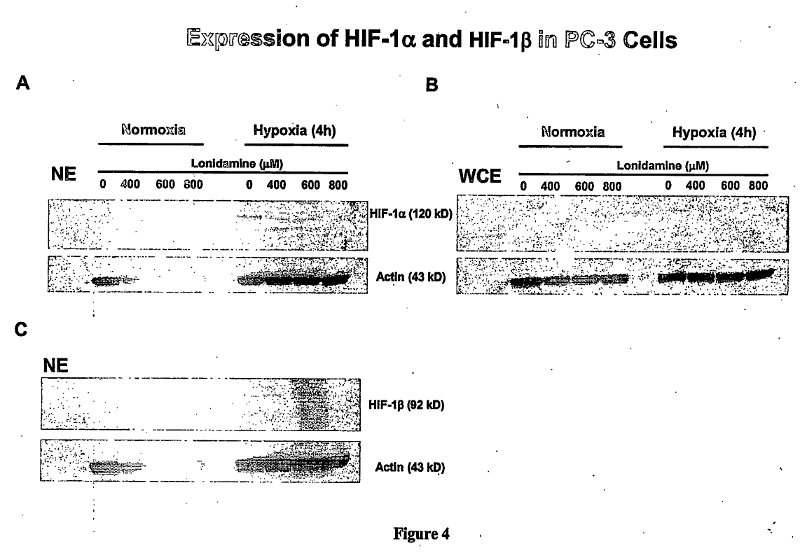

As shown in FIGS. 3 and 4, lonidamine treatment reduced the level of HIF-1α protein detected in nuclear (NE) and whole-cell extract (WCE) preparations. The inhibition was dose-dependent, and observed under normoxic (PC3 cells only) and hypoxic conditions (LNCaP cells and PC3 cells). The lonidamine effect was specific to HIF-1α subunit and, except at 800 μM concentration, had no detectable inhibition under the conditions tested on the protein levels of actin, caspase 3, NF-κB, or IκBα. Lonidamine has, however, been reported to inhibit protein synthesis generally (see Floridi et al., supra), and the results presented herein should not be construed as definitive evidence that lonidamine is a specific inhibitor of HIF-1α or that lonidamine's therapeutic effect in the treatment of BPH is in whole or in part due to its inhibitory effect on the accumulation of HIF-1α in any cell type.

Methods: Cells were plated at a density of 5×10⁵ cells into a dish, and then maintained in 37° C. incubator (5% CO₂) for 2 days. Prior to the assay, cells were rinsed twice with pre-warmed (37° C.) RPMI-1640 Medium (ATCC No. 30-2001; 10 mM HEPES; 1 mM sodium pyruvate; 2 mM L-glutamine; 4500 mg glucose/L; 1500 mg sodium bicarbonate/L). Cells were incubated with 2 ml of culture medium in the absence or presence of lonidamine at different concentrations for 4 hours at 37° C. either under normoxia or hypoxia (oxygen level <0.1%). At the end of the incubation, the dish was placed on ice, and the cells were washed rapidly twice with cold PBS buffer (4° C.). For nuclear extracts, cells were lysed with buffer A (10 mM Tris, pH7.5; 1.5 mM MgCl₂; 10 mM KCl and protease inhibitors) and buffer C (0.5 M NaCl; 20 mM Tris pH7.5; 1.5 mM MgCl₂; 20% glycerol and protease inhibitors), sequentially. The protease inhibitors used in the experiments were a cocktail of five protease inhibitors (500 mM AEBSF-HCl, 1 mg/ml Aprotinin, 1 mM E-64, 500 mM EDTA and 1 mM Leupeptin; Calbiochem NO 539131). For whole cell lysate, cells were lysed with 150 mM NaCl; 10 mM Tris ph7.5; 10 mM EDTA; 1% Triton X-100; 0.5% Deoxycholate, and protease inhibitors. The protein concentration was measured using a Bio-Rad protein assay. Equal amounts of protein were loaded on a SDS-PAGE gel. After transferring of the sample to PVDF membrane, the membrane was blocked with TBST containing 5% non-fat milk overnight at 4° C. Subsequently, the membrane was incubated with primary antibodies (HIF-1α, HIF-1β, and actin) and alkaline phosphatase-conjugated secondary antibody, for two hours each incubation. To detect the expression of caspase 3, NF-κB, P65 and IκBα, the membrane was blocked with TBST-containing 5% non-fat milk for 1 h at room temperature, and the proteins were detected by incubation with the corresponding antibodies overnight at 4° C. and with the alkaline phosphatase-conjugated secondary antibody for 1 h. The specific protein was detected using a colorimetric substrate, and the intensity of each protein was quantified using an NIH image system.

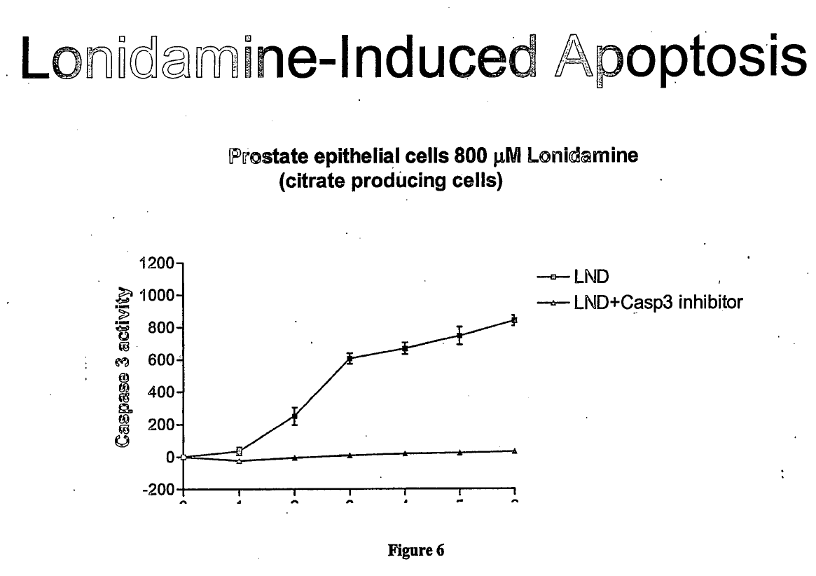

In separate experiments carried out generally as above, the effect of 0-600 μM lonidamine on expression of HIF-1α and other proteins was determined in LNCaP whole cell extracts (FIG. 6) or nuclear extracts (FIG. 7) from cells cultured under hypoxic conditions.

### Example 2

### Lonidamine Induces Apoptosis in Citrate-Producing Cells

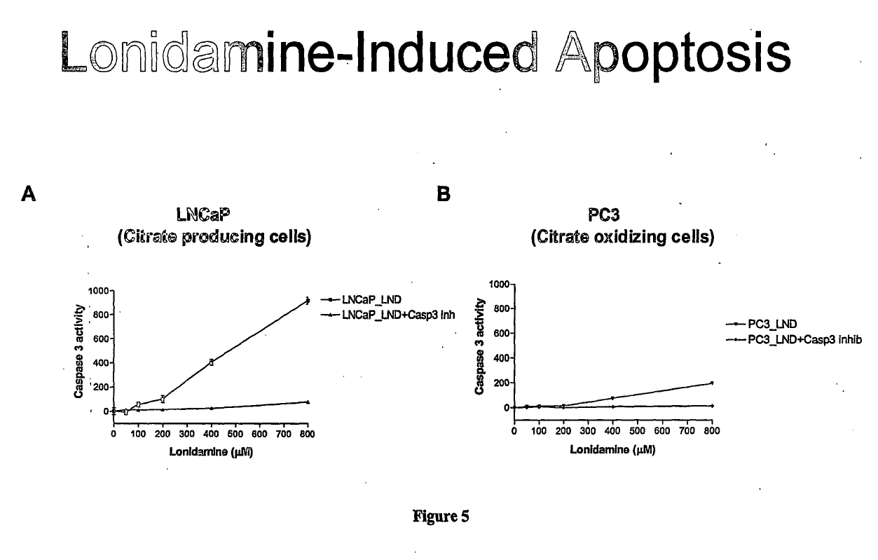

To determine whether apoptosis occurs in cells treated with lonidamine, the effect of lonidamine on cells producing citrate (LNCaP) and cells oxidizing citrate (PC3) was assessed. As shown in FIG. 4, lonidamine induced activation of caspase 3 in citrate-producing cells (LNCaP) to a much greater extent than in citrate-oxidizing cells (PC3). The activation of caspase3 is a time-dependent process (FIG. 5).

The effect of lonidamine was also examined in primary cultures of prostate epithelial cells (which accumulate citrate) and prostate stromal cells, which do not accumulate citrate. As shown in FIG. 5, lonidamine induced apoptosis only in prostate epithelial cells in a dose-dependent manner. In contrast, induction of apoptosis was not observed in prostate stromal cells after treatment with lonidamine.

Methods:

Immunoblotting: Immunoblotting was carried out as described in Example 2. To detect the expression of caspase 3, the membrane was blocked with TBST containing 5% non-fat milk for 1 h at room temperature, and caspase 3 protein was detected by incubation with caspase 3 antibody overnight at 4° C. and with the alkaline phosphatase-conjugated secondary antibody for 1 h. The specific protein was detected using colorimetric substrate, and the intensity of each protein was quantified using an NIH image system.

Primary Cell Cultures: Primary cultures of human prostate epithelial cells (Cambrex No CC-2555) and human prostate stromal cells (Cambrex No CC-2508) were obtained from Cambrex Bio Science Rockland, Inc. (191 Thomaston Street, Rockland, Me. 04841).

Apoptosis assay: Cells were plated at a density of 2×10⁴ cells per well in a 96 well plate, and then maintained in a 37° C. incubator (5% CO₂) for 16 h. Lonidamine was added into each well at different concentrations, and then incubated for 6 h at 37° C. To assess the caspase 3 activity, the homogeneous buffer and caspase 3 substrate (Promega No G7791; Promega Corporation, 2800 Woods Hollow Road, Madison Wis. USA 53711) were added into each well in the presence or absence of caspase 3 inhibitor (Promega No G5961). The fluorescence intensity of cleaved substrate was determined using a fluorescence plate reader at excitation 485 nm and emission 530 nm.

### 9. References Cited

Ansari et al., 1998, “Long-Term Sequelae of Tolnidamine on Male Reproduction And General Body Metabolism in Rabbits” Contraception 57:271-279.

Ansel et al., 1999, in Pharmaceutical Dosage Forms and Drug Delivery Systems, 7th ed., Lippincott Williams & Wilkins, Philadelphia, pp. 1-562.

Avigad and Englard, 1974, “5-Keto-D-Fructose. 8. Synthesis of 5-Keto-D-Fructose 1,6-Bisphosphate and Some of its Properties” Biochem. Biophys. Acta 343(2):330-340.

Barry et al., 1992, “Symptom Index For Benign Prostatic Hyperplasia” J Urol. 148:1549-1557.

Barry et al., 2001, “Measuring the Symptoms And Health Impact of Benign Prostatic Hyperplasia And Its Treatments” BENIGN PROSTATIC HYPERPLASIA (5TH INTERNATIONAL CONSULTATION ON BPH). Health Publication, Ltd.

Barry et al., 2003, “Benign Prostatic Hyperplasic” in SCIENTIFIC AMERICAN MEDICINE, Dale and Federman Eds., WebMD Inc.

Bisswanger, 1981, “Substrate Specificity of the Pyruvate Dehydrogenase Complex From Escherichia Coli” J. Biol. Chem. 256(2):815-822.

Bisswanger, 1980, “Fluoropyruvate: A Potent Inhibitor of the Bacterial And The Mammalian Pyruvate Dehydrogenase Complex” Biochem. Biophys. Res. Commun. 95(2):513-519.

Burger, 1991, “Isosterism and Bioisosterism in Drug Design” A. Prog. Drug Res. 37:287-371.

Cheng et al., 2001, “Two New Male Contraceptives Exert Their Effects By Depleting Germ Cells Prematurely From the Testis” Biol Reprod. 65:449-461.

Chun et al., 2001, “Inhibitory Effect of YC-1 on The Hypoxic Induction of Erythropoietin And Vascular Endothelial Growth Factor in Hep3B Cells” Biochem. Pharmacol. 61(8):947-954.

Colombo, 1975, “Interaction of Inhibitors With Muscle Phosphofructokinase” J. Biol. Chem. 250(24):9404-9412.

Corsi et al., 1976, “1-Halobenzyl-1H-Indazole-3-Carboxylic Acids. A New Class of Antispermatogenic Agents”, Journal of Medicinal Chemistry 19:778-83.

Costello & Franklin, 2000, “The Intermediary Metabolism of The Prostate: A Key to Understanding The Pathogenesis And Progression of Prostate Malignancy” Oncology 59:269-282.

Costello et al., 1999, “Citrate in the Diagnosis of Prostate Cancer” Prostate 38:237-245.

Costello et al., 2000, “Zinc Causes a Shift Toward Citrate at Equilbrium of The m-Aconitase Reaction of Prostate Mitochondria” J. Inorganic Biochemistry 78:161-165.

Costello and Franklin, 1997, “Citrate Metabolism of Normal And Malignant Prostate Epithelial Cells” Urology 50(1):3-12.

Crane and Sols, 1954, “The Non-Competitive Inhibition of Brain Hexokinase by Glucose-6-Phosphate and Related Compounds” J. Biol. Chem. 210(2):597-606.

Fanciulli et al., 1996, “Effect of The Antitumor Drug lonidamine on Glucose Metabolism of Adriamycin-Sensitive And -Resistant Human Breast Cancer Cells” Oncology Research 3:111-120.

Floridi et al., 1981, “Effect of lonidamine on The Energy Metabolism of Ehrlich Ascites Tumor Cells” Cancer Res. 41:4661-4666.

Floridi et al., 1985, “Effect of lonidamine on Protein Synthesis in Neoplastic Cells” Exp. Mol. Path. 42: 293-305.

Franklin et al; 1995, “Regulation of citrate metabolism by androgen in the LNCaP human prostate carcinoma cell line.” Endocrine 3:603-607

Furuta and Hashimoto, 1982, “Pyruvate Dehydrogenase Complex From Pigeon Breast Muscle” Methods in Enzymology 89(Part D):414-420.

Gatto et al., 2002, “Recent Studies on lonidamine, The Lead Compound of The Antispermatogenic Indazol-Carboxylic Acids” Contraception 65:277-278.

Grima et al., 2001, “Reversible Inhibition of Spermatogenesis in Rats Using a New Male Contraceptive, 1-(2,4-Dichlorobenzyl)-Indazole-3-Carbohydrazide” Biol Reprod. 64:1500-1508.

Hagen et al., 2003, “Redistribution of Intracellular Oxygen in Hypoxia by Nitric Oxide: Effect on HIF1” Science 302:1975-1978.

Hanson et al., 1970, “Inhibition fo Phosphofructokinase by Quinone Methide And Alpha-Methyelene Lactone Tumor Inhibitors” Science 168(929):378-380.

Heywood et al., 1981, “Toxicological Studies on 1-Substituted-Indazole-3-Carboxylic Acids” Chemotherapy 27:91-97.

Jay et al., 1990, “Metabolic Stability of 3-O-Methyl-D-Glucose in Brain And Other Tissues” J. Neurochem. 55(3):989-1000.

Jeyaraj et al., 2000, “Effects of Long-Term Administration of Androgens And Estrogen on Rhesus Monkey Prostate: Possible Induction of Benign Prostatic Hyperplasia” J Androl. 21:833-841.

Johnson et al., 1982, “Inhibition of Hexokinase and Protein Kinase Activities of Tumor Cells by a Chloromethyl Ketone Derivative of Lactic Acid” Biochemistry 21:2984-2989.

Kaplan, 2000 “Correspondence re: M. Fanciulli et al., Energy Metabolism of Human LoVo Colon Carcinoma Cells: Correlation to Drug Resistance And Influence of lonidamine.” Clin Cancer Res. 6:4166-4167.

Gunter, 1982, Methods in Enzymology, 90:103-10, 1982

Kurhanewicz et al., 1991, “³¹P Spectroscopy of The Human Prostate Gland in Vivo Using a Transrectal Probe” Magnetic Resonance in Medicine 22:404-413.

Kurhanewicz et al., 2000, “The Prostate: MR Imaging and Spectroscopy. Present and Future” Radiol Clin North Am 38:115-138.

Lee et al., 1998, “Chronology and Urodynamic Characterization of Micturition in Neurohormonally Induced Experimental Prostate Growth in the Rat” Neurourol Urodyn. 17:55-69.

Liu et al., 1990, “Synthesis and Study of (Z)-3-Chlorophosphoenolpyruvate” Arch. Biochem. Biophys. 277:143-148.

LobI et al., 1981, “Effects of Lonidamine (AF 1890) and Its Analogues on Follicle-Stimulating Hormone, Luteinizing Hormone, Testosterone And Rat Androgen Binding Protein Concentrations in The Rat and Rhesus Monkey” Chemotherapy 27:61-76.

Lohiya et al., 1981, “Antispermatogenic Effects of Tolnidamine in Langur (Presbytis entellus)” Contraception 43:485-496.

Lowe and Perham, 1984, “Bromopyruvate as an Active-Site-Directed Inhibitor of the Pyruvate Dehydrogenase Multienzyme Complex From Escherichia Coli” Biochemistry 23(1):91-97.

Machado de Domenech and Sols, 1980, “Specificity of Hexokinases Towards Some Uncommon Substrates And Inhibitors” FEBS Letters 119(1):174-176.

Mansour and Colman, 1978, “Affinity Labeling of AMP-ADP Sites in Heart Phosphofructokinase by 5-p-Fluorosulfonylbenzoyl Adenosine” Biochem. Biophys. Res. Commun. 81:1370-1376.

Mariotti et al., 1982, “Collagen And Cellular Proliferation in Spontaneous Canine Benign Prostatic Hypertrophy” J Urol. 127:795-797.

Marshall, 1979. “Solid Oral Dosage Forms,” MODERN PHARMACEUTICS, Vol. 7, (Banker and Rhodes, editors), pp. 359-427.

McCune et al., 1989, “Aurintricarboxylic Acid is a Potent Inhibitor of Phosphofructokinase” Biochem. J. 259:925-927.

Mendelsohn, 1991, “Principles of Neoplasia” in HARRISON'S PRINCIPLES OF INTERNAL MEDICINE, Wilson ed., McGraw-Hill, New York, p. 1576.

Narayan and Kurhanewicz, 1992, “Magnetic Resonance Spectroscopy in Prostate Disease: Diagnostic Possibilities And Future Developments.” Prostate Suppl. 4:43-50.

O'Leary et al., 1981, “1-Hydroxycyclopropane Carboxylic Acid Phosphate: A Potent Inhibitor of Enzymes Metabolizing Phosphoenolpyruvate” Biochem. Biophys. Res. Commun. 100(3):1320-1325.

Patani and LaVoie, 1996, “Bioisosterism: A Rational Approach in Drug Design” Chem. Rev. 96:3147-3176.

Rose and O'Connell, 1969, “Inactivation and Labeling of Triose Phosphate Isomerase and Enolase by Glycidol Phosphate” J. Biol. Chem. 244(23):6548-6557.

Scopes and Stoter, 1982, “Purification of All Glycolytic Enzymes From One Muscle Extract” Methods in Enzymology 90(Part E):479-490.

Silvestrini et al., 1984, Prog. Med. Chem. 21, G. P. Ellis and G. B. West, eds., Elsevier Science Publishers, Amsterdam, p. 111-135.

Silvestrini, 1981, “Basic and Applied Research n the Study of Indazole Carboxylic Acids” Chemotherapy 27:9-20.

Sols and Crane, 1954, “Substrate Specificity of Brain Hexokinase” J. Biol. Chem. 210(2):581-595.

Spring and Wold, 1971, “Studies on Two High-Affinity Enolase Inhibitors. Reaction With Enolases” Biochemistry 10(25):4655-4660.

Thomas et al., 1990, “¹H MR Spectroscopy of Normal And Malignant Human Prostates in Vivo.” Journal of Magnetic Resonance 87:610-619.

Waymack et al., 1979, “The Effect of Pyruvate Transport Inhibitors on the Regulation of Pyruvate Dehydrogenase in the Perfused Rat Heart” Arch. Biochem. Biophys. 194(1):258-264.

Wirsching and O'Leary, 1985, “(E)-3-Cyanophosphoenolpyruvate, A New Inhibitor of Phosphoenolpyruvate-Dependent Enzymes” Biochemistry 24(26):7602-7606.

US 2003/0087961 “Therapeutics for Cancer Using 3-Bromopyruvate and Other Selective Inhibitors of ATP Production”.

US 2003/0072814 “Topical Pharmaceutical Composition for the Treatment of Warts”.

US 2002/137801 “Methods and Compositions to Treat Conditions Associated With Neovascularization”.

US 2002/0077300 “Screening Method for Cancer Therapeutics and Stable Antitumor Drug”.

US 2002/0035071 “Mimicking the Metabolic Effects of Caloric Restriction by Administration of Glucose Antimetabolites”.

U.S. Pat. No. 3,895,026 “Substituted 1-benzyl-1H-indazole-3-carboxylic Acids and derivatives thereof”.

U.S. Pat. No. 4,381,298 “Inductive Torque Transmitter With Stationary Field Winding”.

U.S. Pat. No. 5,643,883 “Glucose-6-Phosphate Uptake Inhibitors and Novel Uses Thereof”.

U.S. Pat. No. 5,652,273 “Reduction of Hair Growth”.

U.S. Pat. No. 5,824,665 “Reduction of Hair Growth”.

U.S. Pat. No. 6,001,865 “3-substituted 1-benzyl-1H-indazole derivatives as antifertility agents”.

U.S. Pat. No. 6,114,397 “Gossypol for the Treatment of Cancer”.

U.S. Pat. No. 6,146,658 “Prodrugs, their preparation and use as pharmaceuticals.”

U.S. Pat. No. 6,183,948 “Methods to Identify Compounds Affecting Mitochondria”.

U.S. Pat. No. 6,218,435 “Reduction of Hair Growth”.

U.S. Pat. No. 6,479,251 “Colorimetric Test for Agents That Induce Mitochondrial Dysfunction”.

U.S. Pat. No. 6,670,330 “Cancer Chemotherapy With 2-Deoxy-D-Glucose”.

WO 01/82926 “Manipulation of Oxidative Phosphorylation for Hypersensitizing Tumor Cells to Glycolytic Inhibitors”.

WO 02/097053 “Small Molecule Antagonists of BCL2 Family Proteins”.

WO 02/47673 “Methods and Compositions to Treat Conditions Associated With Neovascularization”.

Although the present invention has been described in detail with reference to specific embodiments, those of skill in the art will recognize that modifications and improvements are within the scope and spirit of the invention, as set forth in the claims which follow. All publications and patent documents cited herein are incorporated herein by reference as if each such publication or document was specifically and individually indicated to be incorporated herein by reference. Citation of publications and patent documents (patents, published patent applications, and unpublished patent applications) is not intended as an admission that any such document is pertinent prior art, nor does it constitute any admission as to the contents or date of the same. The invention having now been described by way of written description and example, those of skill in the art will recognize that the invention can be practiced in a variety of embodiments and that the foregoing description and examples are for purposes of illustration and not limitation of the following claims.

1. A method for treating benign prostatic hypertrophy (BPH) comprising administering a therapeutically effective amount of an energolytic agent (EA) to a human subject in need of such treatment, wherein the energolytic agent is an agent that interferes with energy metabolism in prostate epithelial cells.

2-3. (canceled)

4. A method for prophylaxis of BPH comprising administering a prophylactically effective amount of an energolytic agent (EA) to a human subject, wherein the energolytic agent is an agent that interferes with energy metabolism in prostate epithelial cells.

5. The method of claim 1 wherein the energolytic agent is selected from the group consisting of 2-deoxyglucose, 3-bromopyruvate, oxamate, iodoacetate, and apoptolidin.

6. (canceled)

7. The method of claim 1 wherein the subject is neither diagnosed with nor under treatment for cancer.

8. (canceled)

9. The method of claim 1 wherein the subject has a serum PSA less than about 10 ng/ml.

10. (canceled)

11. The method of claim 1 wherein said energolytic agent is administered in combination with an other treatment for BPH.

12. (canceled)

13. The method of claim 1, wherein the energolytic agent is administered at least once daily for at least five days.

14. The method of claim 1 wherein, when compared to a baseline prior to the initiation of treatment, the subject's:
a) AUASI or IPSS score is decreased by at least 3 points, optionally by at least about 5 points;
b) prostate size has decreased by at least about 20%, optionally at least about 40%; and/or
c) serum PSA levels have decreased by at least about 20%, optionally at least about 40%,
when determined on or after 60 days after the initiation of treatment.

15. A method for treating BPH comprising (a) diagnosing BPH in a patient, (b) administering an energolytic agent to the patient and (c) determining whether one or more manifestations of BPH are reduced in said patient.

16. A method for treating BPH comprising (a) administering an energolytic agent to a patient diagnosed with BPH and (b) determining whether one or more manifestations of BPH are reduced in said patient.

17-20. (canceled)

21. A method for determining the usefulness of a compound for treatment of BPH comprising
a) contacting a citrate-producing cell with the compound
b) contacting a citrate-oxidizing cell with the compound
c) detecting a differential effect of said contacting on said citrate-producing cell compared to said citrate-oxidizing cell, wherein a differential effect indicates that the agent may be useful for treatment of BPH.

22. The method of claim 21 wherein the cells are derived from prostate.

23. The method of claim 22 wherein the cells are human.

24. The method of claim 22 wherein the citrate-producing cells and citrate-oxidizing cells are cells cultured under hypoxic conditions.

25. The method of claim 21 wherein the differential effect is induction of apoptosis that is greater in citrate-producing cells compared to citrate-oxidizing cells.

26. The method of claim 21 wherein the citrate-producing cells are a primary culture of human prostate ephthelial cells and the citrate-oxidizing cells are a primary culture of human prostate stromal cells

27. The method of claim 21 wherein the citrate-producing cells and citrate-oxidizing cells are established cell lines.

28. The method of claim 21 wherein the citrate-producing cells are LNCaP cells and the citrate-oxidizing cells are PC-3 cells.

29. A method for determining the usefulness of a compound for treatment of BPH comprising
(a) contacting a citrate-producing cell cultured under conditions of hypoxia with the compound; and
(b) identifying a compound as useful for treatment of BPH if the contacting results in a dose-dependent reduction in HIF-1alpha expression (measured in the nuclear fraction) of at least about 2-fold.

30. The method of claim 29 wherein the citrate-producing cell is an LNCaP
